(19) United States
(12) Patent Application Publication
(10) Pub. No.: US 2001/0024795 A1
(43) Pub. Date: Sep. 27, 2001

(54) Immunoassay technique using multispecific molecules

(76) Inventors: Ban-an Khaw, Milton, MA (US); Jagat Narula, Rosemont, PA (US)

Correspondence Address: PENNIE AND EDMONDS, 1155 AVENUE OF THE AMERICAS, NEW YORK, NY, 100362711

(21) Appl. No.: 09/727,421

(22) Filed: Dec. 1, 2000

Related U.S. Application Data
(63) Continuation-in-part of application No. 09/380,168, filed on Oct. 6, 1999.
(60) Provisional application No. 60/039,111, filed on Feb. 26, 1997.

(51) Int. Cl. C12Q 1/70; G01N 33/53

(52) U.S. Cl. 435/7.1; 435/5

(57) ABSTRACT

The present invention relates to compositions and methods for detecting very low concentrations of a molecule in a mixture. The detection of the molecule comprises the steps of first contacting a sample with a multispecific molecule capable of binding at least two molecules including the molecule to be detected, wherein the molecule to be detected is bound by the multispecific molecule thereby forming a complex, and second contacting the complex with a second, different molecule which is linked via a polymer to multiple detection signaling molecules. The invention may also be practiced by administration of the multispecific molecule in vivo, to a host for the molecule to be detected, either with or without the bound polymer probe and thereafter, respectively, either detecting the signaling molecule on the probe, or administering the probe and allowing it to bind the multispecific molecule, followed by detection of the signaling molecule on the probe.

### CROSS REFERENCE TO RELATED APPLICATIONS

This application is a continuation-in-part of U.S. patent application Ser. No. 09/380,168, filed Oct. 6, 1999, which is the national stage application of International Publication No. WO 98/38513, filed Feb. 25, 1998, which claims the benefit of priority to U.S. Provisional Application No. 60/039,111 filed Feb. 26, 1997, the contents of each of which are incorporated herein by reference in their entirety.

### FIELD OF THE INVENTION

The present invention relates to compositions and methods for detecting very low concentrations of a molecule in a mixture. The detection of the molecule comprises the steps of first contacting a sample with a multispecific molecule capable of binding at least two molecules including the molecule to be detected, wherein the molecule to be detected is bound by the multispecific molecule thereby forming a complex, and second contacting the complex with a second, different molecule which is linked via a polymer to multiple detection signaling molecules.

### BACKGROUND OF THE INVENTION

The sensitivity of a detection assay is, in part, limited by the strength of signal and the number of signaling molecules available, for example on an antibody in a direct binding assay, or alternatively, on the probe compound in a competitive inhibition assay. In immunoassays, by increasing the number of signaling molecules, such as radioisotopes, horseradish peroxidase or alkaline phophatase, in order to increase the assay sensitivity, the signaling molecules frequently denature and thus inactivate the antibody or alternatively denature the antigen also resulting in decreased detection sensitivity. Thus, in these traditional assays, additional antibody or probe must be added in order to obtain greater signal sensitivity with limited success.

The delayed detection of a pathogen or disease state often leads to a greater risk of permanent damage to tissues or even increased risk of mortality. In many diseases there are marker molecules which increase in expression in correlation with disease progression. Marker molecules can be antigens associated with or produced by a disease, and are increased in concentration concurrently with an increase in progression of the disease. Thus, the increase in the marker molecule correlates with an increase in pathogenicity, and hence a worsening of the disease condition, e.g., a viral pathogen such as HIV, or a bacterial pathogen such as Salmonella. The diseased organism may also react to a pathogen or pathogenic condition, where the organism starts to produce or increase production of markers that are not normally present or are present in low levels in the organism, e.g., heart attack victims show increased levels of CK-MB, Troponin-T or I.

Thus, it is to the advantage of the diagnostician to identify markers, as soon as possible, when the markers are at a relatively low concentration and the disease has not yet progressed to a more severe state, in order to initiate a proper therapeutic regimen to minimize the risk of mortality or morbidity.

For most detection assays, there is a lag time for the compound of interest to reach a high enough concentration in the serum to become detectable for diagnostic purposes. In the case of heart attacks, there is a delay of 4-6 hours from the onset of chest pain until the diagnostic detection of CK-MB, Troponin-T or I is possible. Myoglobin is detectable earlier, but its specificity is low.

Thus, there is a need for an assay which could detect very minute levels or increased sensitivity to increased levels of these and other indicator molecules in the patient's blood, for example, at an earlier point in time, so that therapeutic intervention could be started earlier and thereby bring about greater myocardial salvage.

### SUMMARY OF THE INVENTION

The present invention relates to compositions and methods for detecting very low concentrations of a molecule in a mixture. The detection of the molecule comprises the steps of contacting a sample with a multispecific molecule capable of binding at least two molecules including the molecule to be detected, wherein the molecule to be detected is bound by the multispecific molecule thereby forming a complex, followed by contacting said complex with a second, different molecule which is linked via a polymer to multiple detection signaling molecules.

In a specific embodiment, the invention contemplates a method for detecting very low concentrations of antigens of interest in a sample comprising: contacting the sample with a multispecific molecule for a sufficient time to allow a complex to form between the multispecific molecule and the antigen, and then contacting the complex with a labeled detection probe to form an antigen-multispecific molecule-probe complex whereby the complex can be detected at very low concentrations, thereby resulting in the detection of the antigen of interest in the sample.

The invention encompasses an immunoassay method for detecting antigens of interest in a sample comprising: contacting said sample first with a multispecific molecule and a labeled detection probe to form an antigen-molecule-probe complex; detecting the antigen-molecule-probe complex, thereby detecting the antigens of interest in the sample.

The invention encompasses a method of imaging an antigen bearing structure in a patient, comprising: administering to the patient multispecific molecule and a detection probe; allowing an antigen-molecule-probe to form; detecting the antigen-molecule-probe, thereby imaging the antigen bearing structure. The imaging probes can be radiolabeled or paramagnetically labeled or fluorescent labeled, such that they are capable of being detected using conventional imaging techniques.

The invention encompasses a kit for detecting an antigen of interest in a sample, comprising: a labeled detection probe, a multispecific molecule, with instructions for using the kit to detect an antigen of interest in a body.

In a preferred embodiment, the multispecific molecule is a bispecific molecule, and most preferably is a bispecific antibody, or fragments thereof, that is either chemically crosslinked or is expressed recombinantly as a fusion protein.

In a preferred embodiment, the polymer is a chemically synthesized polylysine molecule.

In a preferred embodiment, the labeling molecule is horseradish peroxidase. In another preferred embodiment, the labeling molecule is I¹²⁵.

The invention also encompasses a two part detectable complex comprising a multispecific molecule and a polymeric detection probe, wherein the probe comprises at least 6 labeling molecules and preferably more than 6 detection molecules.

### BRIEF DESCRIPTION OF THE DRAWINGS

FIG. 1a shows a standard ELISA according to the prior art.

FIG. 1b depicts an immunoassay according the invention.

FIG. 2 is a graph showing competitive inhibition curves using standard ELISA (R11D10), bispecific antibody complex with standard secondary antibody for signal production (BiMAb (Ab-HRP)), and the method according to the invention (BiMAb (PL-DTPA-HRP)).

FIG. 3 is a graph demonstrates the increasing detection capacity of an ELISA with the R11D10 antibody in a bispecific antibody-antigen complex using the polymer probes according to the invention (DTPA-PL-HRP6, DTPA-PL-HRP12, DTPA-PL-HRP18, DTPA-PL-HRP24 and DTPA-PL-HRP30).

### DETAILED DESCRIPTION OF THE INVENTION

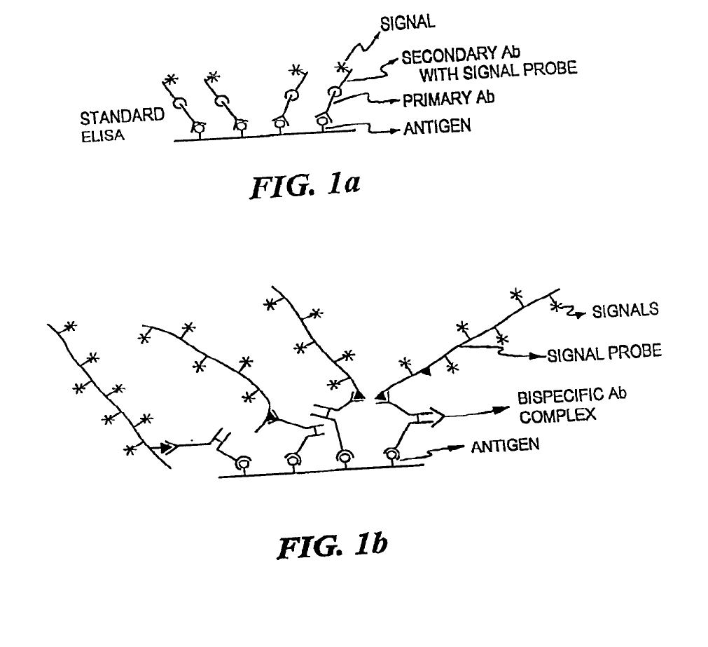

The instant invention relates to compositions and methods to enhance the sensitivity of a detection assay over traditional assay methods (FIG. 1a). In particular, the invention encompasses compositions and methods which significantly improve detection of molecules by at least 100,000 times over standard immunoassays without losing specificity. This improvement is achieved, for example, by the use of a bispecific molecule complex and a unique detection signal probe capable of being recognized by the bispecific molecule complex (FIG. 1b).

The instant invention is directed to the development of a new approach to the use of multispecific molecules in immunoassays. In particular, the multispecific molecule comprises at least one binding region specific for the antigen of interest to be detected and another binding region specific for a labeled detection probe which is not present in the sample. The two binding regions can be chemically or genetically linked.

In a preferred embodiment, the multispecific molecule is a bispecific antibody. In another preferred embodiment, the bispecific molecule is a bispecific antibody that is not formed by chemically cross-linking two antibodies. The bispecific antibody has a first variable region having specificity to a molecule to be detected and the second variable region having specificity to a second molecule, different from the molecule to be detected, which is linked to a polymer, wherein the polymer is attached to at least 2, more preferably 3 or more detection molecules.

The term “antibodies or variable regions having specificity to a molecule” as used herein refers to antibodies or fragments thereof that specifically bind to a molecule which is a polypeptide antigen or a fragment of a molecule and do not non-specifically bind to other antigens. Antibodies or fragments that specifically bind to a molecule or fragment thereof may have cross-reactivity with other antigens. Preferably, antibodies or fragments that specifically bind to a molecule polypeptide or fragment thereof do not cross-react with other antigens. Antibodies or fragments that specifically bind to a molecule polypeptide can be identified, for example, by immunoassays or other techniques known to those of skill in the art.

Previously, many antibodies had to react with the antigen of interest to develop sufficient signal intensity for detection. Only a few molecules of the detection probe of this invention are needed to provide a signal. The probe can be labeled using radioactivity, paramagnetism, chemical color producing enzymes, or flourescent probes can be attached to its backbone.

The term “multispecific molecule” includes any agent, e.g., a protein, peptide, protein or peptide complex, or chemical entity, which has more than two different binding specificities which bind to, or interact with (a) an antigen of interest, and (b) a binding moiety of at least one detection probe. The term “bispecific molecule” includes any agent, e.g., a protein, peptide, protein or peptide complex, or other chemical entity which has two different binding specificities which bind to, or interact with (a) an antigen of interest and (b) the binding moiety of at least one detection probe. Accordingly, the invention includes, but is not limited to, heteroantibodies, bispecific, trispecific, tetraspecific, and other multispecific molecules which bind to antigens of interest and to detection probes.

The term “sample” includes mixtures which may contain the antigen. Preferably, the samples are obtained from living sources, such as animals, e.g., mammals, e.g., dogs, cats, horses, pigs, bears, cattle, sheep, goats, rabbits, mice, rats, squirrels, primates, e.g., gorillas, chimpanzees, monkeys, or, preferably, humans. The sample can be a body fluid (e.g., blood, plasma, saliva, urine, etc.), a body tissue sample such as a biopsy (e.g., neuronal, muscle, or organ tissue), a mixture, or, in certain embodiments, an entire organism. Other samples include air, water, plants or vegetation, soil, minerals and like materials.

The term “label” includes radiolabels, flourescent labels, enzymatic labels, paramagnetic labels, or any other moiety which can be used to detect the complex. Examples of radiolabels include, but are not limited to I¹²⁵, ³⁵S, ¹⁴C, ³H, Tc⁹⁹M, Mg⁵² or Fe. The radiolabels can be detected either directly or indirectly, and the radioisotope detected by direct counting of radioemmission or by scintillation counting. Alternatively, the probes can be enzymatically labeled with, for example, horseradish peroxidase, alkaline phosphatase, or luciferase, and the enzymatic label detected by determination of conversion of an appropriate substrate to product.

The term “detection probe” includes probes which are capable of interacting with the multispecific molecule and forming molecule-antigen-probe complexes. In one embodiment, the detection probe is labeled with 7 or more labels, 8 or more labels, 9 or more labels, 10 or more labels, 11 or more labels, 12 or more labels, 13 or more labels, 14 or more labels, 15 or more labels, 16 or more labels, 17 or more labels, 18 or more labels, 19 or more labels, 20 or more labels, 21 or more labels, 22 or more labels, 23 or more labels, 24 or more labels, 25 or more labels, 26 or more labels, 27 or more labels, 28 or more labels, 29 or more labels, 30 or more labels, 31 or more labels, 32 or more labels, 33 or more labels, 34 or more labels, 35 or more labels, 40 or more labels, 45 or more labels, or 50 or more labels. In one embodiment, the detection probe is comprised of a polymer, e.g., polypeptide, e.g., polylysine backbone. The detection probe comprises a binding moiety which interacts with the multispecific molecule. In a further embodiment, the detection probe is initially a positively charged polymer that is converted to neutral or negative charge in the final probe. For example, the detection probe can be a polymer comprised at least in part of positively charged monomers, e.g., lysine and/or arginine. In further embodiments, the detection probe may also comprise drugs or other moieties which can advantageously be delivered to the cell using the methods of the invention.

The term “binding moiety” refers to moieties which bind to the detection probe such that the multispecific molecule can bind to the detection probe. In one embodiment, the binding moiety is a protein or another moiety which is foreign to the sample. The binding moiety should be selected such that ti interacts with the sample and detection probe with high specificity. Examples of binding moieties include, but are not limited to DTPA (diethylenetriaminepentaacetate), EDTA (ethylenediaminetetraacetate), anti-DTPA antibodies, and anti-EDTA antibodies and other antigen and antibody pairs not generally found with in the sample.

The term “antibody conjugate” includes two or more antibodies linked together to form a multispecific molecule. It includes heteroantibodies, which refer to two or more antibodies (e.g., monoclonal, recombinant or bispecific antibodies), antibody binding fragments (e.g., Fab), derivatives therefrom, or antigen binding regions linked together, at least two of which have different specificities. These different specificities may advantageously include, for example, a binding specificity for the binding moiety of the detection probe, and a binding specificity for an antigen of interest, e.g., an antigen of a tumor cell.

The term “monoclonal antibody” includes antibodies which display a single binding specificity and affinity for a particular epitope. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant antibody” includes all antibodies that are prepared, expressed, created or isolated by recombinant means, such as antibodies isolated from a transgenic animal (e.g., a mouse) immunoglobulin genes; antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

The term “specific binding” refers to antibody binding to a predetermined antigen. Typically, the antibody binds with an affinity of at least about 1×10⁷ M⁻¹, and binds to the predetermined antigen with an affinity that is at least two-times greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen”.

In a further embodiment, by using the methods of the present invention, less than 2×10⁻¹⁶ mole of antigen can be detected. For example, conventional methods typically can detect about 2×10⁻¹⁴ mole of antigen in a sample. There is a great need for more sensitive assays to detect lower concentrations of antigens for the early diagnosis of conditions related to the antigens. In another embodiment, the method of the invention can detect antigens at a concentration of about 2×10⁻¹⁶ mole or less, about 1×10⁻¹⁶ mole or less, about 6×10⁻¹⁷ mole or less, about 2×10⁻¹⁷ mole or less, about 1×10⁻¹⁷ mole or less, about 6×10⁻¹⁸ mole or less, about 2×10⁻¹⁸ mole or less, about 1×10⁻¹⁸ mole or less, about 6×10⁻¹⁹ mole or less, about 2×10⁻¹⁹ mole or less, about 1×10⁻¹⁹ mole or less, about 6×10⁻²⁰ mole or less, about 2×10⁻²⁰ mole or less, about 1×10⁻²⁰ mole or less, about 6×10⁻²¹ mole or less, about 2×10⁻²¹ mole or less, about 1×10⁻²¹ mole or less, about 6×10⁻²² mole or less, about 2×10⁻²² mole or less, about 1×10⁻²² mole or less, about 6×10⁻²³ mole or less, about 2×10⁻²³ mole or less, about 1×10⁻²³ mole or less, about 6×10⁻²⁴ mole or less, about 2×10⁻²⁴ mole or less, about 1×10⁻²⁴ mole or less, or about 1×10⁻²⁵ mole or less of an antigen. Methods of testing the assay for sensitivity can be found in the Examples. The increased sensitivity of the assay is a surprising and unexpected result.

Therefore, the immunoassay sensitivity can be amplified by at least 100,000 times compared to conventional immunoassays or immunosandwich assays. Because early detection of many pathological states, such as acute myocardial infarction and cancer, are limited by the relatively lesser sensitivity of conventional immunoassays to detect minute elevations of the pathologically associated compounds methods and compounds of the invention will enable diagnosis of disease states at a much earlier time than previous assays which may allow for better therapeutic intervention.

Another advantage of the method of the invention is the versatility for adaptation to any antibody. For example, in a preferred embodiment, the method could be adapted to detect Troponin-I or T by using the antibody specific for Troponin I or T attached to a second antibody such as the antibodies shown herein, that recognizes the detector probe. If higher sensitivity is necessary, the detection probe could be generated to carry higher number of signal compounds.

Furthermore, the detection probe can include any kind of signal compound, such as radioisotopes or flourescent or paramagnetic linked signal compounds. All previously existing ELISA radioimmunoassays, dipstick assays for cancer pregnancy serum enzymes and probes and any assays utilizing antibodies could be modified according to the method of the invention to provide enhanced sensitivity. In addition, in vivo application to enhance target signal by using the method of the invention is also possible.

### Bispecific Antibodies

In a preferred embodiment, the multispecific molecules are bispecific antibodies which are produced by fusion of two hybridoma cell lines (Hybrid Hybridoma). Fusion of two hybridomas results in up to ten different antibody products. The ten different antibodies result from association of the different heavy and light chain genes produced. However, the bispecific antibody is readily purified in quantities sufficient for use as an immunotherapeutic using standard column chromatography, cell sorting or immuno-purification schemes as described below.

In yet another embodiment, bispecific antibodies are produced by introduction of antibody genes by transfection into a system to recombinantly express bispecific antibodies in, for example fibroblasts, hybridomas, myelomas, insect cells, or any protein expression system.

In yet another embodiment, bispecific antibodies are produced by isolation of the individual monoclonal antibodies, breaking of disulfide linkages of each specific antibody and subsequent recombination of antibody heavy and light chain polypeptides in vitro (see, for example Arathoon et al., WO 98/50431).

### Antibodies

Immunologically active fragments of immunoglobulin molecules include F(ab) and F(ab′)2 fragments which can be generated by treating the antibody with an enzyme such as pepsin or papain. Examples of methods of generating and expressing immunologically active fragments of antibodies can be found in U.S. Pat. No. 5,648,237 which is incorporated herein by reference in its entirety.

The immunoglobulin molecules are encoded by genes which include the kappa, lambda, alpha, gamma, delta, epsilon and mu constant regions, as well as a myriad of immunoglobulin variable regions. Light chains are classified as either kappa or lambda. Light chains comprise a variable light (V_{L}) and a constant light (C_{L}) domain. Heavy chains are classified as gamma, mu, alpha, delta, or epsilon, which in turn define the immunoglobulin classes IgG, IgM, IgA, IgD and IgE, respectively. Heavy chains comprise variable heavy (V_{H}), constant heavy 1 (CH1), hinge, constant heavy 2 (CH2), and constant heavy 3 (CH3) domains. The IgG heavy chains are further sub-classified based on their sequence variation, and the subclasses are designated IgG1, IgG2, IgG3 and IgG4.

Antibodies can be further broken down into two pairs of a light and heavy domain. The paired V_{L} and V_{H} domains each comprise a series of seven subdomains: framework region 1 (FR1), complementarity determining region 1 (CDR1), framework region 2 (FR2), complementarity determining region 2 (CDR2), framework region 3 (FR3), complementarity determining region 3 (CDR3), framework region 4 (FR4) which constitute the antibody-antigen recognition domain.

A chimeric antibody may be made by splicing the genes from a monoclonal antibody of appropriate antigen specificity together with genes from a second human antibody of appropriate biologic activity. More particularly, the chimeric antibody may be made by splicing the genes encoding the variable regions of an antibody together with the constant region genes from a second antibody molecule. This method is used in generating a humanized monoclonal antibody wherein the complementarity determining regions are mouse, and the framework regions are human (U.S. Pat. Nos. 4,816,567, 4,816,397, 5,693,762; 5,585,089; 5,565,332 and 5,821,337 which are incorporated herein by reference in their entirety).

A bispecific antibody suitable for use in the present invention may be obtained from natural sources or produced by hybridoma, recombinant or chemical synthetic methods, including modification of constant region functions by genetic engineering techniques (U.S. Pat. No. 5,624,821). The bispecific antibody of the present invention may be of any isotype, e.g., IgG, IgM, IgE, IgD or IgA.

Antibodies exist for example, as intact immunoglobulins or can be cleaved into a number of well-characterized fragments produced by digestion with various peptidases, such as papain or pepsin. Pepsin digests an antibody below the disulfide linkages in the hinge region to produce a F(ab)′₂ fragment of the antibody which is a dimer of the Fab composed of a light chain joined to a V_{H}-C_{H}1 by a disulfide bond. The F(ab)′₂ may be reduced under mild conditions to break the disulfide linkage in the hinge region thereby converting the F(ab)′₂ dimer to a Fab′ monomer. The Fab′ monomer is essentially an Fab with part of the hinge region. See Paul, ed., 1993, Fundamental Immunology, Third Edition (New York: Raven Press), for a detailed description of epitopes, antibodies and antibody fragments. One of skill in the art will recognize that such Fab′ fragments may be synthesized de novo either chemically or using recombinant DNA technology. Thus, as used herein, the term antibody fragments includes antibody fragments produced by the modification of whole antibodies or those synthesized de novo.

As used herein, an antibody can also be a single-chain antibody (scFv), which generally comprises a fusion polypeptide consisting of a variable domain of a light chain fused via a polypeptide linker to the variable domain of a heavy chain.

Methods for preparing bi- and multispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858, each of which is incorporated herein by reference in their entirety.

### Antibody Production

Antibodies can be prepared by immunizing a suitable subject with an antigen as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see generally Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., 1975, Nature, 256:495, or may be made by recombinant DNA methods (U.S. Pat. No. 4,816,567). The term “monoclonal antibody” as used herein also indicates that the antibody is an immunoglobulin.

In the hybridoma method of generating monoclonal antibodies, a mouse or other appropriate host animal, such as a hamster, is immunized as hereinabove described to elicit lymphocytes that produce or are capable of producing antibodies that will specifically bind to the protein used for immunization (see generally, U.S. Pat. No. 5,914,112, which is incorporated herein by reference in its entirety.)

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immuno-absorbent assay (ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal antibody-secreting hybridomas, a monoclonal antibody directed against a pathogen or detection molecule polypeptide of the invention can be identified and isolated by screening a recombinant combinatorial immunoglobulin library (e.g., an antibody phage display library) with the antigen of interest. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.)

Humanized antibodies are antibody molecules from non-human species having one or more complementarity determining regions (CDRs) from the non-human species and a framework region from a human immunoglobulin molecule. (see e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Riechmann et al. (1988, Nature, 332:323; antibodies against hepatitis B in Cole et al. (1991, Proc. Natl. Acad. Sci. USA 88:2869); as well as against viral antigens-respiratory syncitial virus in Tempest et al. (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

Completely human antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of an immunogen.

Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif. (see, for example, U.S. Pat. No. 5,985,615)) and Medarex, Inc. (Princeton, N.J.), can be engaged to provide human antibodies directed against a selected antigen using technology similar to that described above.

Completely human antibodies which recognize and bind a selected epitope can be generated using a technique referred to as “guided selection.” In this approach a selected non-human monoclonal antibody, e.g., a mouse antibody, is used to guide the selection of a completely human antibody recognizing the same epitope (Jespers et al. (1994) antigen Bio/technology 12:899-903).

A pre-existing antibody directed against a pathogen can be used to isolate additional antigens of the pathogen by standard techniques, such as affinity chromatography or immunoprecipitation for use as immunogens. Moreover, such an antibody can be used to detect the protein (e.g., in a cellular lysate or cell supernatant) in order to evaluate the abundance and pattern of expression of the pathogen. The antibodies can also be used diagnostically to monitor pathogen levels in tissue as part of a clinical testing procedure, e.g., determine the efficacy of a given treatment regimen. Detection can be facilitated by coupling the antibody to a detectable substance. Examples of detectable substances include, but are not limited to various enzymes, prosthetic groups, fluorescent materials, luminescent materials, bioluminescent materials, and radioactive materials. Examples of suitable enzymes include, but are not limited to horseradish peroxidase, alkaline phosphatase, betagalactosidase, or acetylcholinesterase; examples of suitable prosthetic group complexes include, but are not limited to streptavidin/biotin and avidin/biotin; examples of suitable fluorescent materials include, but are not limited to umbelliferone, fluorescein, fluorescein isothiocyanate, rhodamine, dichlorotriazinylamine fluorescein, dansyl chloride or phycoerythrin; an example of a luminescent material includes, but is not limited to luminol; examples of bioluminescent materials include, but are not limited to luciferase, luciferin, and aequorin; and examples of suitable radioactive material include, but are not limited to I¹²⁵, ³⁵S, ¹⁴C, ³H, Tc⁹⁹M, Mg⁵² or Fe.

Antibodies that are commercially available can be purchased and used to generate bispecific antibodies, e.g., from ATCC®. In a preferred embodiment of the invention, the antibody is produced by a commercially available hybridoma cell line. In a more preferred embodiment, the hybridoma secretes a human antibody.

### Bispecific Antibody Production and Purification

Production of full length bispecific antibodies is based on the coexpression of two immunoglobulin heavy chain-light chain pairs in a single hybridoma cell line, where two sets of antibody encoding genes encode for antibodies having different antigen specificities (Milstein et al., 1983, Nature, 305:537-539). Because of the random assortment of immunoglobulin heavy and light chains, these hybridomas (i.e., ‘quadromas’) produce a potential mixture of 10 different antibody molecules, of which only one has the correct bispecific structure (L₁H₁H₂L₂). Purification of the correct molecule, which is usually done by affinity chromatography steps, is rather cumbersome, and the product yields are low. Alternative purification procedures are disclosed in WO 93/08829, published May 13, 1993, and in Traunecker et al., 1991, EMBO J., 10:3655-3659.

The invention thus provides method of producing a bispecific immunoglobulinsecreting cell comprising the steps of: (a) fusing a first cell expressing an immunoglobulin which binds to a marker molecule with a second cell expressing an immunoglobulin which binds to a detection molecule; and (b) selecting for cells that express a bispecific immunoglobulin that comprises a first binding domain which binds to a Marker molecule, and a second binding domain which binds to a detection molecule.

In a specific embodiment, a bispecific immunoglobulin of the invention is produced recombinantly (see, e.g., U.S. Pat. No. 4,816,397 dated Mar. 28, 1989 by Boss).

Thus, the invention provides a method for producing a bispecific molecule comprising a first binding domain which binds a marker molecule and a second binding domain which binds a detection molecule in a cell, comprising the steps of: (a) transforming a cell with a one or more first DNA sequences encoding at least the first binding domain and a one or more second DNA sequences encoding at least the second binding domain; and (b) expressing said first DNA sequences and said second DNA sequences so that said first and second binding domains are produced as separate molecules which assemble together in said transformed cell, whereby a bispecific molecule is formed that (i) binds a marker molecule, and (ii) binds the detection molecule.

The invention also provides a method for producing a bispecific molecule comprising a first binding domain which binds a Marker molecule and a second binding domain which binds a detection molecule in a cell, comprising the steps of: (a) transforming a first cell with one or more first DNA sequences encoding at least the first binding domain; (b) transforming a second cell with one or more second DNA sequences encoding at least the second binding domain; (c) expressing said first DNA sequences and said second DNA sequences so that said first and second binding domains are produced separately; (d) isolating said first and second binding domains; and (e) combining said first and second binding domains in vitro to form a bispecific molecule that binds the marker molecule and binds the detection molecule.

The invention further provides a cell transformed with a first nucleotide sequence encoding a first binding domain and a second nucleotide sequence encoding a second binding domain, wherein when expressed in the cell, the two binding domains associate together to form a bispecific molecule, wherein the first binding domain binds a marker molecule, and the second binding domain binds a detection molecule.

In one embodiment, the bispecific antibodies are produced recombinantly, whereby nucleotides which encode antibody variable domains with the desired binding specificities (antibody-antigen combining sites) are fused to nucleotides which encode immunoglobulin constant domain sequences. The fusion preferably is with an immunoglobulin heavy chain constant domain, comprising at least part of the hinge, CH2, and CH3 regions. It is preferred to also have the first heavy-chain constant region (CH1) containing an amino acid residue with a free thiol group so that a disulfide bond may be allowed to form during the translation of the protein in the hybridoma, between the variable domain and heavy chain (see, Arathoon et al., WO 98/50431).

DNAs encoding the immunoglobulin heavy chain fusions and, if desired, the immunoglobulin light chain, are inserted into separate expression vectors, and are co-transfected into a suitable host organism. This provides for the ability to adjust the proportions of each of the three polypeptide fragments in unequal ratios of the three polypeptide chains, thus providing optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm fused to the constant CH2 and CH3 domains, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details of generating bispecific antibodies see, for example, Suresh et al., 1986, Methods in Enzymology, 121:210.

In another preferred embodiment, a bispecific antibody fragment can be prepared by any one of the following non-limiting examples. For example, Fab′ fragments recovered from E. coli can be chemically coupled in vitro to form antibodies. See, Shalaby et al., 1992, J. Exp. Med., 175:217-225. Various techniques exist for making and isolating bispecific antibody fragments directly from recombinant cell culture. For example, heterodimers have been produced using leucine zippers (Kostelny et al., 1992, J. Immunol. 148:1547-1553). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers.

The “diabody” technology described by Hollinger et al., (1993, Proc. Natl. Acad. Sci. USA, 90:6444-6448) reported an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites (i.e., bispecific). In a similar protocol, Gruber et al. report the production of bispecific antibody fragments using only single-chain Fv (scFv) dimers (1994, J. Immunol., 152:5368).

### Purification/Isolation or Bispecific Antibodies

In a preferred embodiment, bispecific antibodies secreted from the antibody secreting cells are isolated by ion exchange chromatography (See Section 6.2). Non-limiting examples of columns suitable for isolation of the bispecific antibodies of the invention include, but are not limited to DEAE, Hydroxylapatite, Calcium Phosphate (Staerz and Bevan, 1986, Proc. Natl. Acad. Sci., 83:1453-1457).

In another preferred embodiment, properly fused cells (hybrid-hybridomas) are selected using fluorescent activated cell sorting (FACS). For example, before fusion, each hybridoma is grown in media with label, such as fluorescein isothiocyanate (FITC) or tetramethyl rhodamine isothiocyanate (TRITC). The first hybridoma is grown with a marker that is different from the second hybridoma. The cells are then fused by conventional methods and the bispecific antibody producing cells are identified and selected using FACS by measuring the fluorescent color of the cells (see Koolwijk et al., 1988, Hybridoma 7:217-225; or Karawajew et al., 1987, J. Immun. Methods, 96:265-270).

In another embodiment, bispecific antibodies secreted from the antibody secreting cells are isolated by three-step successive affinity chromatography (Corvalan and Smith, 1987, Cancer Immunol. Immunother., 24:127-132): the first column is made of protein A bound to a solid matrix, where the Fc portion of the antibody binds protein A, and wherein the antibodies bind the column; followed by a second column that utilizes Marker molecule binding to a solid matrix which assays for Marker molecule binding via a first variable domain; and followed by a third column that utilizes specific binding of an antigen of interest bound by a second variable domain.

In yet another embodiment, bispecific antibodies secreted from the antibody secreting cells are isolated by isoelectric focusing of antibodies. The skilled artisan will recognize that any method of purifying proteins using size or affinity will be suitable in the present invention.

### Other Bispecific Molecules

Other bispecific molecules are within the scope of the invention and can be made using techniques well known in the art of molecular biology. In particular, cloning of DNAs can be performed as taught by Current Protocols in Molecular Biology, Ausubel et al., eds., John Wiley & Sons, 1992. Expression of recombinant proteins is also well known in the art.

In one embodiment, the bispecific molecule of the invention is a single molecule (preferably a polypeptide) which consists essentially of, or alternatively comprises, a first binding domain (BD1) bound to the amino terminus of a CH2 and CH3 portion of an immunoglobulin heavy chain (Fc) bound to a second binding domain (BD2) at the Fc domain's carboxy terminus. In another embodiment, the CH2 domain and the CH3 domain positions are present in reverse order. One of the binding domains binds a Marker molecule, and the other of the binding domains binds a detection molecule. The binding domains can individually be a scFv (i.e., a V_{L} fused via a polypeptide linker to a V_{H}) or a receptor or ligand or binding domain thereof, or other binding partner, with the desired specificity. For example, the binding domain that binds the detection molecule can be a cellular receptor for a virus (e.g., CD4 and/or a chemokine receptor, which bind to HIV), or a receptor for a bacteria (e.g., polymyxin or multimers thereof which bind to Gram-negative bacteria), or a cellular receptor or ligand for a drug or other molecule (e.g., ≢ domain of the IgE receptor which binds IgE, to treat or prevent allergic reactions), or a receptor for an autoantibody (e.g., acetylcholine receptor, for treating or preventing myasthenia gravis).

In an embodiment where a binding domain is not a polypeptide or is not otherwise readily expressed as a fusion protein with the other portions of the bispecific molecule, such binding domain can be cross-linked to the rest of the bispecific molecule. For example, polymyxin can be cross-linked to a fusion polypeptide comprising CH₂CH₃ and the binding domain that binds a Marker molecule.

In another embodiment, the bispecific molecule of the invention is a dimeric molecule consisting of a first molecule (preferably a polypeptide) consisting essentially of, or comprising, a BD1 bound to the amino terminus of an immunoglobulin Fc domain (a hinge region, a CH2 domain and a CH3 domain), and a second molecule (preferably a polypeptide), consisting essentially of, or comprising, a Fc domain with a BD2 domain bound to the Fc domain's carboxy terminus, wherein the Fc domains of the first and second polypeptides are complementary to and can associate with each other. BD1 and BD2 are as described above.

In a specific embodiment, one or both of the monomers of the bispecific molecule (preferably a polypeptide) consists essentially of, or comprises, a variable light chain domain (VL) and constant light chain domain (CL) followed by a linker molecule (of any structure/sequence) bound to the amino terminus of a variable heavy chain domain, followed by a CH1 domain, a hinge region, a CH2 domain, and a CH3 domain.

In another embodiment, the bispecific molecule of the invention is a molecule comprising two separate scFv with specificity for two separate antigens (one of which is the marker molecule, the other of which is the detection molecule). The bispecific molecule (preferably polypeptide) consists essentially of, or comprises, a first scFv domain bound to a CH2 domain, followed by a CH3 domain, and a second scFv domain.

In another embodiment, the multispecific molecule of the invention is a bispecific molecule consisting essentially of, or comprising, two variable regions with specificity for two separate antigens. The molecule (preferably polypeptide) consists essentially of, or comprises, a first variable heavy chain domain bound to a variable light chain domain, followed by a CH2 domain, a CH3 domain, a variable heavy chain domain, and a variable light chain domain.

Furthermore, the invention also encompasses rearrangement of the position of any of the individual components of the bispecific molecules, wherein the bispecific molecule retains the ability to detect molecules. For example, the position of two binding domains may be switched for the bispecific molecule. Alternatively, the positions of the CH2 and CH3 domains may be switched. Further, the invention contemplates that the domains may be further rearranged into different positions relative to one another, while retaining its functional properties, i.e., binding to a marker molecule and binding to a detection molecule.

Moreover, as will be clear from the discussion above, the binding domains described above preferably, but need not be, polypeptides (including peptides). Moreover, the binding domains can provide the desired binding specificity via covalent or noncovalent linkage to the appropriate structure that mediates binding.

The foregoing bispecific molecules are preferably obtained by recombinant expression of genetically engineering nucleic acid constructs encoding the bispecific molecules, which can be made using methods well known in the art, and/or extracellular crosslinking methodology.

### Antigens in a Sample

In one embodiment, the invention encompasses an immunoassay method for detecting an antigen of interest in a sample. The method includes contacting the sample with a multispecific molecule and a labeled detection probe to form an antigen-molecule-probe complex, and then detecting the complex. In a preferred embodiment, the multispecific molecule is a bispecific antibody in which one antibody recognizes the antigen of interest and the second antibody recognizes a second antigen which is bound to a polymer.

The term “antigen” includes any molecule that can be detected using the immunoassay of the invention. The term includes, for example, small molecules present in body fluids such as drugs, toxins, autoantibodies, autoantigens, proteins, carbohydrates, nucleic acids and other molecules. Examples of antigens potential present in the serum of a subject include, but are not limited to drugs, such as barbiturates, tricyclic antidepressants, and Digitalis, tumor antigens such as antigens associated with breast, prostate, brain, liver, kidney, colon, pancreatic, stomach, or lung cancer, viral antigens (e.g., antigens associated with or produced by HIV, influenza or other viruses), bacterial antigens, for example in systemic bacterial infections, hormones (e.g., thyroid stimulating hormone (TSH), human growth hormones, progesterone, testosterone, human chorionic gonadotrophin (hCG)), plasma proteins (e.g., a fibrin degradation product (FDP), a C-reactive protein (CRP), a carcinoembryonic protein, α-fetoprotein (AFP), carcinoembryonic antigen (CEA)), plaque antigens, haptens (e.g., angiotensin I, vasopressin, somatostatin, atrial natriuretic hormone, endoserine, luteinizing hormone releasing hormone (LH-RH), kassinin or other peptides), steroids (e.g., cortisol), and cytokines such as interleukin-1 (IL-1), interferon-α, interferon-β, interferon-γ, interleukin-2 (IL-2), interleukin-4 (IL-4), interleukin-6 (IL-6) interleukin-7 (IL-7), interleukin-12 (IL-12), interleukin-15 (IL-15), B7, CD28, or other members of the Ig superfamily.

Additional antigens that can be detected by the use of methods and compositions of the invention include, but are not limited to antigens associated with or produced by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsachie virus, mumps virus, measles virus, rubella virus, polio virus, human immunodeficiency virus type I (HIV-I), and human immunodeficiency virus type II (HIV-II), any picornaviridae, enteroviruses, caliciviridae, any of the Norwalk group of viruses, togaviruses, such as Dengue virus, alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, Epstein-Barr virus, human herpesvirus-6, cercopithecine herpes virus 1 (B virus), and poxviruses.

Bacterial antigens that can be detected by the use of methods and compositions of the invention include, but are not limited to antigens associated with or produced by Mycobacteria rickettsia, Mycoplasma, Neisseria spp. (e.g., Neisseria menigitidis and Neisseria gonorrhoeae), Legionella, Vibrio cholerae, Streptococci, such as Streptococcus pneumoniae, Corynebacteria diphtheriae, Clostridium tetani, Bordetella pertussis, Haemophilus spp. (e.g., influenzae), Chlamydia spp., enterotoxigenic Escherichia coli, etc.

Protozoal antigens that can be detected by the use of methods and compositions of the invention include, but are not limited to antigens associated with or produced by plasmodia, eimeria, Leishmania, and trypanosoma. Additional antigens include pollutants, toxins, noxious chemicals, forensic material and the like.

### Imaging Methods Using Multispecific Molecules

In another embodiment, the invention also encompasses methods for imaging an antigen bearing structure in a patient, e.g., a mammal, e.g., a human (e.g., a human suffering from a tumor). The method includes administering to the patient an multispecific molecule and a detection probe, allowing an antigen-molecule-probe to form, and detecting the antigen-molecule-probe. Positron emission tomography (“PET”) and single photon emission computed tomography (“SPECT”) can also be used.

The term “antigen bearing structure” includes tumors and other organs or structures in the body which may exhibit unusual antigens. In one embodiment, the antigen bearing structure is a tumor, e.g., a breast, prostate, brain, liver, kidney, colon, pancreatic, stomach, or lung tumor.

In a further embodiment, the multispecific molecule is immuno-multispecific molecule, such as, for example, a bispecific antibody. Other multispecific molecules include conjugation of receptors, oligonucleotides and the like. In a further embodiment, the detection probe and the multispecific molecule are administered in a pharmaceutically acceptable manner, so that both the molecule and the probe are able to perform their intended functions, e.g., label and image the antigen bearing structure in the patient. In yet a further embodiment, the multispecific molecule and the probe are administered in pharmaceutically acceptable carriers.

For example, the multispecific molecules and the probes of the invention are useful for the localization and in vivo imaging of antigen bearing structures, e.g., tumors, for specific treatment of diseased cells, e.g. site-directed delivery of cytotoxins, immuno-modulators or other pharmaceutically active molecules where local concentration of the active agent is an important factor, or the like. For in vivo imaging, the detection probe can be, for example, radiolabelled or conjugated with a metal chelate complexed with a radionuclide, e.g. iodine, yttrium, technetium, or the like, and radioscanning techniques can be used to detect antigen bearing structures, such as, for example, primary and metastatic tumors. For example, the detection probe and the multispecific molecule are injected e.g. intravenously and the patient scanned with a gamma imager at regular intervals. Structures expressing antigens of interest will interact more with the multispecific molecules and the detection probes than other tissue and will be clearly recognized by the gamma imaging camera. Examples of radiolabels include ¹³¹I for radioscanning. For biocidal activity in the treatment of antigen bearing structures, e.g., tumors, the detection probes can be conjugated to cytostatic or cytotoxic substances, e.g. lectins (e.g., ricin, abrin), diphtheria toxin A, and the like.

In another embodiment, the detection probe further comprises at least one drug moiety. The drug moiety can be targeted to the cells using the antigens. The drug moiety can be chosen such that it is effective against the structure displaying the antigen. For example, an anti-tumor drug, such as paclitaxel or doxorubicin, may be used when the antigen of interest is a tumor antigen.

### Pharmaceutical Compositions

In one embodiment, the multispecific molecules and probes of the invention may be administered to a patient in order to perform imaging assays. When the multispecific molecules and probes of the invention are administered to a human, they are advantageously administered as a pharmaceutical composition comprising a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., multispecific molecule, may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition of the present invention can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred per cent, this amount will range from about 0.01 per cent to about ninety-nine percent of active ingredient, preferably from about 0.1 per cent to about 70 per cent, most preferably from about 1 per cent to about 30 per cent.

Formulations of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Examples of suitable aqueous and nonaqueous carriers which may be employed in the pharmaceutical compositions of the invention include, but are not limited to water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given alone or as a pharmaceutical composition containing, for example, 0.01 to 99.5% (more preferably, 0.1 to 90%) of active ingredient in combination with a pharmaceutically acceptable carrier.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a compositions of the invention will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic compositions may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include, but are not limited to: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multichamber compartments; and U.S. Pat. No. 4,475,196, which discloses an osmotic drug delivery system. These patents are incorporated herein by reference. Many other such implants, delivery systems, and modules are known to those skilled in the art.

The composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as manitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound may be orally administered, for example, with an inert diluent or an assimilable edible carrier.

### Kits of the Invention

The invention also encompasses a kit for detecting an antigen of interest in a sample. The kit includes a labeled detection probe, a multispecific molecule, instructions for using the kit to detect an antigen of interest in a body sample, and a container.

In one embodiment, the multispecific molecule is a recombinantly expressed bispecific antibody.

In another embodiment, the sample is blood, saliva, or plasma from a patient, e.g., a human patient.

In another embodiment, the kit is capable of detecting antigens at concentrations of about 2×10⁻¹⁶ M or less, about 1×10⁻¹⁶ M or less, about 6×10⁻¹⁷ M or less, about 2×10⁻¹⁷ M or less, about 1×10⁻¹⁷ M or less, about 6×10⁻¹⁸ M or less, about 2×10⁻¹⁸ M or less, about 1×10⁻¹⁸ M or less, about 6×10⁻¹⁹ M or less, about 2×10⁻¹⁹ M or less, about 1×10⁻¹⁹ M or less, about 6×10−²⁰ M or less, about 2×10⁻²⁰ M or less, about 1×10⁻²⁰ M or less, about 6×10⁻²¹ M or less, about 2×10⁻²¹ M or less, about 1×10⁻²¹ M or less, about 6×10⁻²² M or less, about 2×10⁻²² M or less, about 1×10⁻²² M or less, about 6×10⁻²³ M or less, about 2×10⁻²³ M or less, about 1×10⁻²³ M or less, about 6×1⁻²⁴ M or less, about 2×10⁻²⁴ M or less, about 1×10⁻²⁴ M or less, or about 1×10⁻²⁵ M or less.

In another embodiment, the instructions or the packaging of the kit indicate that the kit is capable of detecting antigens at concentrations of 2×10⁻¹⁶ M or less, about 1×10⁻¹⁶ M or less, about 6×10⁻¹⁷ M or less, about 2×10⁻¹⁷ M or less, about 1×10⁻¹⁷ M or less, about 6×10⁻¹⁸ M or less, about 2×10⁻¹⁸ M or less, about 1×10⁻¹⁸ M or less, about 6×10⁻¹⁹ M or less, about 2×10⁻¹⁹ M or less, about 1×10⁻¹⁹ M or less, about 6×10⁻²⁰ M or less, about 2×10⁻²⁰ M or less, about 1×10⁻²⁰ M or less, about 6×10⁻²¹ M or less, about 2×10⁻²¹ M or less, about 1×10⁻²¹ M or less, about 6×10⁻²² M or less, about 2×10⁻²² M or less, about 1×10⁻²² M or less, about 6×10⁻²³ M or less, about 2×10⁻²³ M or less, about 1×10⁻²³ M or less, about 6×10⁻²⁴ M or less, about 2×10⁻²⁴ M or less, about 1×10⁻²⁴ 10 M or less, or about 1×10⁻²⁵ M or less.

In another embodiment, the invention encompasses a detectable complex comprising a marker antigen of interest, a multispecific molecule, and a detection probe. In one embodiment, the complex is detectable at concentrations of, for example, 2×10⁻¹⁶ M or less, about 1×10⁻¹⁶ M or less, about 6×10⁻¹⁷ M or less, about 2×10⁻¹⁷ M or less, about 1×10⁻¹⁷ M or less, about 6×10⁻¹⁸ M or less, about 2×10⁻¹⁸ M or less, about 1×10⁻¹⁸ M or less, about 6×10⁻¹⁹ M or less, about 2×10⁻¹⁹ M or less, about 1×10⁻¹⁹ M or less, about 6×10⁻²⁰ M or less, about 2×10⁻²⁰ M or less, about 1×10⁻²¹ M or less, about 6×10⁻²¹ M or less, about 2×10⁻²¹ M or less, about 1×10⁻²¹ M or less, about 6×10⁻²² M or less, about 2×10⁻²² M or less, about 1×10⁻²² M or less, about 6×10⁻²³ M or less, about 2×10⁻²³ M or less, about 1×10⁻²³ M or less, about 6×10⁻²⁴ M or less, about 2×10⁻²⁴ M or less, about 1×10⁻²⁴ M or less, or about 1×10⁻²⁵ M or less.

The invention also encompasses a polymer detection probe, which comprises a neutral or positive polymer backbone (e.g., polylysine or polyarginine), about 18 or more labels (e.g., horseradish peroxidase labels), and a specific binding moiety. The specific binding moiety can be DTPA or another antigen, epitope or binding moiety not generally found with in the sample being assayed. The specific binding moiety can be, for example an antibody, e.g., an anti-DTPA antibody.

The present invention is further illustrated by the following examples which should not be construed as further limiting. The contents of all figures and all references, patents and published patent applications cited throughout this application are expressly incorporated herein by reference.

### EXAMPLE 1

Serum immunoassays for intracardiac contractile proteins constitute the mainstay for detection of myocyte necrosis associated with various cardiovascular disorders. However, myosin heavy chain (MHC) fragments can be detected by prior art immunoassays only after 48 hours from the onset of chest pain. To enhance immunodetection of MHC, monoclonal antibodies (mAb) R11D10 or 264-2D7, which are both specific for cardiac MHC, were covalently linked to MAb 4G4-1D5 specific for DTPA. The detection probe consisted of DTPA-modified poly lysine (28:1 molar ratio) covalently linked to horse-radish peroxidase (6 moles/mole polylysine) (PL-DTPA-HRP). Porcine cardiac myosin (PCM, 1 μg/ml) was used to coat the microtiter wells. After overnight incubation and washing, three times, 50 μl each of 5 μg/ml BiMAb or MAb and serial dilutions of PCM (0.001 to 100 μg/ml) or 50 μl of serial dilutions (1/1 to 1/10000) of patient sera pre-incubated for 1 hour at 37° C. After washing, the wells were incubated with goat-antimouse IgG-HRP or PL-DTPA-HRP for two hours. A chromogen, dinitrobenzidine was used to develop the assay. The affinity of BiMAb and R11D10 were the same at 1.5×10⁹ L/mole. The sensitivity of BiMAb was 0.5 μg, whereas that of R11D10 was 0.5 μg (1 μg/ml). BiMAb developed with the conventional goat-antimouse IgG-HRP had a sensitivity of 0.05 μg.

Therefore, BiMAb assay has a 1000 times increase in sensitivity compared to the conventional immunoassay in the sera of 3 heart transplant patients. Using the BiMAb assay, 2.5, 1.25 and 1.3 ng MHC/50 μl serum at 1/1 dilution, were detected.

### EXAMPLE 2

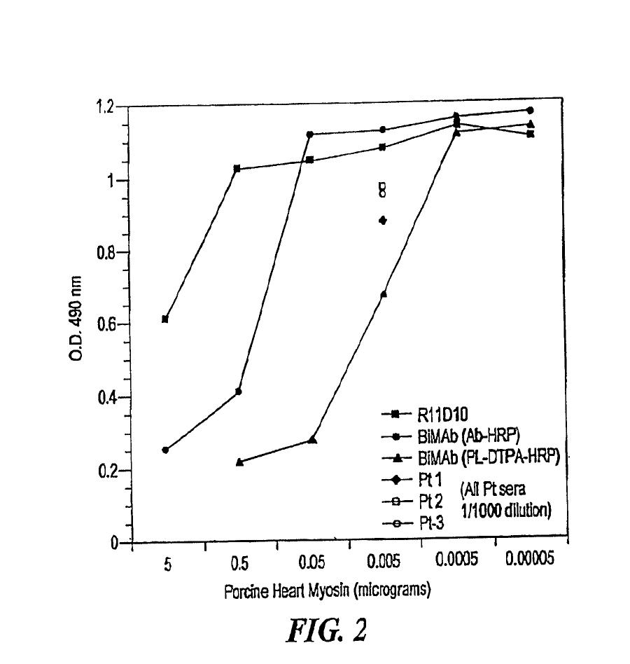

In a subsequent experiment, the DTPA-modified polylysine probe of Example 1, was covalently linked to 12 moles of horse-radish peroxidase per mole of polylysine. The results of the study (FIG. 2) show that the sensitivity of the bispecific assay of the invention (10⁻⁸ to 100 μg/ml) was at least 10,000 times better that the conventional immunoassay (0.1 μg/ml).

### EXAMPLE 3

In vitro assays for diagnosis of acute myocardial infarction typically rely on the detection of the release of soluble intracellular cardiac macromolecules into the circulation. Assays such as CK, CK-MB, Troponin I and T can detect myocardial necrosis as early as 4 to 6 hours after the on-set of chest pain. Earlier detection is not feasible using the prior art assays due to the limit of sensitivity of the assays employed. A polymer-probe-bi specific monoclonal antibody assay with at least 10,000 times increase in sensitivity relative to the conventional ELISA or radioimmunoassay, utilizing myosin heavy chain fragments (MHC-f) and monoclonal antimyosin antibody. Conventional assays can detect MHC-f only 48 hours after the onset of chest pain. Since MHC is an insoluble major contractile protein of the myocaridal cells, its release into the circulation may denote myocardial necrosis. Therefore, an assay which detects MHC-f very early after the onset of chest-pain, should be highly specific for diagnosis of acute myocardial infarction.

In this example, a polymer-probe-bi-specific monoclonal antibody assay (utilizing a murine monoclonal antibody for β cardiac myosin heavy chain) has been used to detect MHC0f in the first blood sample of patients with Q-MI, non-Q-MIs, unstable angina, congestive heart failure, Hiatus Hernia and three normal subjects.

The bispecific antibody (BiSA) was prepared using purified 4G41 D5 anti-DTPA antibody which was modified with SPDP and subsequently tested for activity. The purified R11D10 antimyosin antibody was modified with iminothiolane and its activity was subsequently assessed. The two antibody preparations were then reacted at equal concentrations. The resulting bispecific antibodies were separated from individual unreacted antibodies by Ultro-gel AcA-22 molecular sieve column chromatography. The purified bispecific antibody was confirmed by electrophoresis in by non-reducing SDS-PAGE.

The polymer detection probes consisted of DTPA modified polylysine antigen-molecule-probed with 6, 12, 18, 24 or 30 horse radish peroxidase enzymes per polymer. Residual amino groups were succinylated. The sensitivity of the standard ELISA with R11D10 utilizing the conventional HRP conjugated secondary antibody was 2×10⁻¹⁴ moles of myosin. With 6, 12, and 18 HRP DTPA-polymer probes and BiSA, the sensitivity increase to 2×10⁻¹⁶, 2×1⁻²¹ and 2×10⁻²¹ moles respectively. Using the 6, 12, and 18 HRP-polymer-BiSA sera (straight, 1/10 and 1/100 dilutions tested respectively) from AMI, unstable angina and normal volunteers were assessed. Mean concentrations of β-isomyosin epitopes by all 3 BiSA assays of the admission sera of Q and non-Q wave MIs were 1.9±0.17 and 3.25±0.2 m/ml respectively. The UA patient was 1.93±0.115. Normal volunteer sera were negative. Total Ck was positive only in UA patient's admission serum (319 U/L).

Since the epitope for β-isomyosin generally originates from the fibrous myofilaments of the cardiocyctes its presence in the sera should indicate the occurrence of myocardial autolysis following necrosis. Polymer based BiSA detects minute amounts of the antigen released into the circulation very shortly after the onset of necrosis, thus providing an early and specific diagnosis of AMI.

### EXAMPLE 4

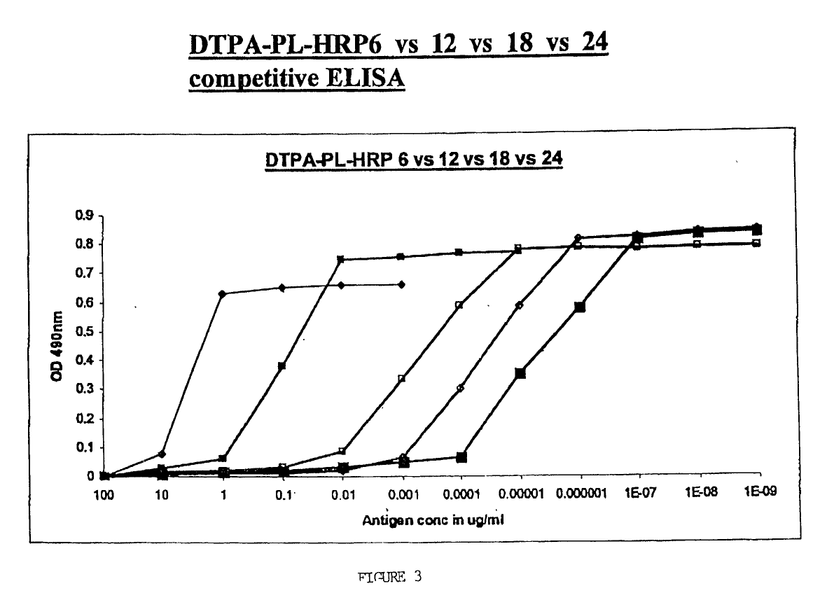

In another subsequent experiment, the DTPA-modified polylysine probe of Example 1, was covalently linked to 18, 24 and 30 moles of horse-radish peroxidase per mole of polylysine. The results of the study (FIG. 3) show that the sensitivity of the bispecific assay of the invention was at least 100,000,000 times better that the conventional immunoassay (0.1 μg/ml).

Equivalents

Those skilled in the art will recognize, or be able to ascertain using no more than routine experimentation, many equivalents of the specific embodiments of the invention described herein. Such equivalents are intended to be encompassed by the following claims.

What is claimed is:

1. A method of detecting an antigen of interest in a sample comprising:
contacting the sample with a multispecific molecule, said multispecific molecule being capable of simultaneously binding the antigen of interest and a labeled detection probe, and being other than two monoclonal antibodies that are chemically cross linked, and allowing an antigen-molecule complex to form;
contacting the sample with a labeled detection probe, wherein said detection probe comprises at least seven moles of a detectable label, for sufficient time to form an antigen-molecule-probe complex; and
detecting the antigen-molecule-probe.

2. The method of claim 1, wherein said antigen of interest is selected from the group consisting of drug antigens, tumor antigens, viral antigens, bacterial antigens, hormones, plasma proteins, plaque antigens, haptens, and steroids.

3. The method of claim 3, wherein said tumor antigen is associated with breast, prostate, brain, liver, kidney, colon, pancreatic, stomach, or lung cancer.

4. The method of claim 3, wherein said viral antigens are associated with hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsachie virus, mumps virus, measles virus, rubella virus, polio virus, human immunodeficiency virus type I (HIV-I), and human immunodeficiency virus type II (HIV-II), picornaviridae, enteroviruses, caliciviridae, Norwalk viruses, Dengue virus, alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, Epstein-Barr virus, human herpesvirus-6, cercopithecine herpes virus 1 (B virus), and poxviruses.

5. The method of claim 3, wherein said hormone is thyroid stimulating hormone (TSR) or human chorionic gonadotrophin (hCG).

6. The method of claim 3, wherein said plasma protein is a fibrin degradation product (FDP), a C-reactive protein (CRP), a carcinoembryonic protein, α-fetoprotein (AFP), or a carcinoembryonic antigen (CEA).

7. The method of claim 3, wherein said hapten is angiotensin I, vasopressin, somatostatin, atrial natriuretic hormone, endoserine, luteinizing hormone releasing hormone (LH-RH), kassinin or other peptides.

8. The method of claim 3, wherein said steroid is progesterone, testosterone, cortisol or another steroid.

9. The method of claim 1, wherein said sample is a sample from a living organism or an inanimate object.

10. The method of claim 9, wherein said living organism is a human patient.

11. The method of claim 10, wherein said sample from a human patient is a tissue, blood, saliva, or plasma sample.

12. The method of claim 1, wherein said assay is conducted in vitro.

13. The method of claim 1, wherein about 2×10⁻¹⁶ mole of the antigen is present in the sample.

14. The method of claim 12, wherein about 2×10⁻¹⁸ mole of the antigen is present in the sample.

15. The method of claim 12, wherein about 2×10⁻²¹ mole of the antigen is present in the sample.

16. The method of claim 1, wherein said detection probe comprises a polymer backbone.

17. The method of claim 16, wherein said polymer backbone is polylysine.

18. The method of claim 1, wherein the detection probe is labeled with a radiolabel.

19. The method of claim 1, wherein the detection probe is labeled with a flourescent label.

20. The method of claim 1, wherein the detection probe is labeled with an enzymatic label.

21. The method of claim 20, wherein said label is horseradish peroxidase.

22. The method of claim 1, wherein the detection probe is paramagnetically labeled.

23. The method of claim 22, wherein said detection probe is labeled with at least 9 labels.

24. The method of claim 22, wherein said detection probe is labeled with at least 12 moles of label.

25. The method of claim 22, wherein said detection probe is labeled with at least 18 moles of label.

26. A method of imaging an antigen bearing structure in a patient, comprising:
administering to the patient a multispecific molecule, said multispecific molecule being other than two monoclonal antibodies that are chemically cross linked, and allowing an antigen-molecule complex to form;
administering to the patient a detection probe, wherein said detection probe comprises at least seven moles of a detectable label, and allowing an antigen-molecule-probe to form; and
detecting the antigen-molecule-probe, thereby imaging the antigen bearing structure.

27. The method of claim 26, wherein said antigen bearing structure is a tumor.

28. The method of claim 26, wherein said tumor is a breast, prostate, brain, liver, kidney, colon, pancreatic, stomach, or lung tumor.

29. The method of claim 26, wherein said patient is a human.

30. The method of claim 26, wherein said assay has a sensitivity of about 2×10⁻¹⁶ per mole of antigen.

31. The method of claim 30, wherein said assay has a sensitivity of about 2×10⁻¹⁸ per mole of antigen.

32. The method of claim 30, wherein said assay has a sensitivity of about 2×10⁻²¹ per mole of antigen.

33. The method of claim 26, wherein said detection probe comprises a polylysine backbone.

34. The method of claim 26, wherein said detection probe is labeled with at least 9 labels.

35. The method of claim 33, wherein said detection probe is labeled with at least 12 labels.

36. The method of claim 33, wherein said detection probe is labeled with at least 18 labels.

37. The method of claim 33, wherein said detection probe is labeled with at least 24 labels.

38. The method of claim 26, wherein said label is a radiolabel.

39. The method of claim 26, wherein said label is paramagnetic.

40. The method of claim 26, wherein said detection probe further comprises a drug moiety.

41. A kit for detecting for detecting an antigen of interest in a sample, comprising:
a labeled polymer detection probe;
a multispecific molecule which is other than two monoclonal antibodies that are chemically cross linked; and
instructions for using the kit to detect an antigen of interest in a body.

42. The kit of claim 41, wherein said sample is a patient's blood, saliva, or plasma.

43. The kit of claim 41, wherein said patient is a human.

44. The kit of claim 41, wherein said detection probe comprises a polylysine backbone.

45. The kit of claim 41, wherein the detection probe is labeled with a radiolabel.

46. The kit of claim 41, wherein the detection probe is labeled with a flourescent label.

47. The kit of claim 41, wherein the detection probe is labeled with an enzymatic label.

48. The kit of claim 47, wherein said label is horseradish peroxidase.

49. The kit of claim 41, wherein said detection probe is labeled with at least 18 labels.

50. The kit of claim 49, wherein said detection probe is labeled with at least 24 labels.

51. A detectable complex comprising an antigen of interest, a multispecific molecule which is other than two monoclonal antibodies that are chemically cross linked, and a detection probe.

52. The detectable complex of claim 51, wherein said complex is detectable at concentrations of 2×10⁻¹⁸ M or less of the antigen.

53. A detection probe comprising a polylysine backbone, about 18 or more horseradish peroxidase labels per mole of probe, and one or more binding moieties.

54. The detection probe of claim 53, wherein said binding moiety is DTPA.

55. A detection probe of claim 54, comprising about 24 or more horseradish peroxidase labels per mole of probe.
